(19) United States
(12) Patent Application Publication
(10) Pub. No.: US 2026/0047943 A1
(43) Pub. Date: Feb. 19, 2026

(54) Motion Tracking With Implant Augments

(71) Applicant: Howmedica Osteonics Corp., Mahwah, NJ (US)

(72) Inventors: Vivienne Clayton, Montville, NJ (US); Jillian Tuliebitz, London (GB)

(21) Appl. No.: 18/804,977

(22) Filed: Aug. 14, 2024

(51) Int. Cl. A61F 2/48; A61F 2/30; A61F 2/38

(52) U.S. Cl. CPC A61F 2/488; A61F 2/3859; A61F 2/389; A61F 2002/3067

(57) ABSTRACT

Disclosed herein are joint implants and methods for tracking joint implant performance. A joint implant according to the present disclosure can include a first augment on a first implant and a second augment on a second implant of a joint. The first augment can include medial and lateral markers. The second augment can include a medial marker reader to identify the medial markers and a lateral marker reader to identify the lateral markers to provide positional data of the first augment with respect to the second augment. The second augment can include a medial load sensor to measure medial load data and a lateral load sensor to measure lateral load data. A processor coupled to the medial marker reader, the lateral marker reader, the medial load sensor, and the lateral load sensor can transmit the positional data, the medial load data and the lateral load data to an external source.

### FIELD OF INVENTION

The present disclosure relates to implants and methods for tracking implant performance, and particularly to joint implants with augments and methods for tracking performance of same.

### BACKGROUND OF THE INVENTION

Effective monitoring of patient recovery following joint replacement surgery is essential for proper rehabilitation. This process primarily involves the assessment of the implant's functionality to identify any issues such as dislocation, wear, malfunction, or breakage. For instance, a polyethylene (PE) tibial insert used in total knee arthroscopy (TKA) operations can develop premature macroscopic failure from excessive loading or mechanical loosening. Timely detection of any suboptimal functioning of the implant, or signs of infection and inflammation at the site of implantation allows the administration of corrective treatments before the implant fails. Furthermore, monitoring data concerning the postoperative range of motion and the load distribution on newly implanted TKA devices can assist in managing the patient's recovery process and determining the need for a replacement implant if necessary.

The current diagnostic methods for assessing implant performance are primarily limited to patient feedback and imaging techniques, such as X-ray fluoroscopy and magnetic resonance imaging (MRI). However, patient feedback may not always be reliable. For example, patients may not perceive gradual implant wear, dislocation, or the onset of an infection. Moreover, these imaging techniques provide limited insights. For instance, X-ray images do not capture details about the patient's range of motion, or the stress exerted on the knee joint in patients recovering from total knee arthroscopy (TKA). Additionally, these methods only offer a momentary glimpse of the implant's condition, lacking the capability to deliver continuous, real-time data on implant performance.

The integration of sensors and associated electronic components into implants is challenging primarily due to the limited space within the implant structure and the need to maintain the implant's integrity and functionality. Implants such as those used in TKA are designed to mimic the biomechanics of natural joints and thus have limited room for additional elements without compromising their primary function. Additionally, these components must not only be unobtrusive but also highly resilient to withstand the rigorous, biomechanical environment of the human body.

Therefore, there exists a need for implants and related methods for tracking implant performance.

### BRIEF SUMMARY OF THE INVENTION

Disclosed herein are joint implants and methods for tracking joint implant performance.

In accordance with an aspect of the present disclosure a joint implant is provided. A joint implant according to this aspect may include a first augment coupled to a first implant of a joint and a second augment coupled to a second implant of the joint. The first augment may include at least one marker. The second implant may contact the first implant. The second augment may include at least one marker reader to detect a position of the marker to identify positional data of the first augment with respect to the second augment, and at least one load sensor to measure load data between the first and second implants. A processor of the second augment may be operatively coupled to the marker reader and the load sensor. The processor may output the positional data and the load data to an external source.

Continuing in accordance with this aspect, the first augment may be a femoral augment and the first implant may be a femoral implant. The second augment may be a tibial augment and the second implant may be a tibial implant. The tibial implant may include a tibial insert and a tibial baseplate. The femoral augment may include a medial femoral augment and a lateral femoral augment. The tibial augment may include a medial tibial augment and a lateral tibial augment.

Continuing in accordance with this aspect, the marker may be a magnet and the marker reader may be a magnetic sensor. The magnetic sensor may be a Hall sensor assembly including at least one Hall sensor. The magnet may be a magnetic track disposed along a surface of the first implant.

Continuing in accordance with this aspect, the positional data may include any of a knee flexion angle, knee varus-valgus rotation, knee internal-external rotation, knee medial-lateral translation, superior-inferior translation, anterior-posterior translation, and time derivatives thereof.

Continuing in accordance with this aspect, the load data may include any of a medial load magnitude, lateral load magnitude, medial load center and lateral load center.

Continuing in accordance with this aspect, the first augment or the second augment may include any of a pH sensor, a temperature sensor and a pressure sensor operatively coupled to the processor.

Continuing in accordance with this aspect, the joint implant may further include a transmitter to transmit the positional data and the load data to an external source. The external source may be any of a tablet, computer, smart phone, and remote workstation.

In accordance with another aspect of the present disclosure, a method for monitoring a joint implant performance is provided. A method according to this aspect, may include the steps of coupling a first augment to a first implant of a joint, the first augment may include at least one magnetic marker, coupling a second augment to a second implant of the joint, the second implant may be configured to contact the first implant, the second augment may include at least one magnetic sensor to detect a magnetic flux density of the magnetic marker, tracking magnetic flux density magnitudes over time using the magnetic sensor, and initiating a warning when a tracked magnetic flux density magnitude is different from a predetermined value.

Continuing in accordance with this aspect, the step of coupling the first augment may include coupling a femoral augment to a femoral implant. The step of coupling the second augment may include coupling a tibial augment to a tibial implant. The femoral augment may include a medial femoral augment and a lateral femoral augment. The tibial augment may include a medial tibial augment and a lateral tibial augment.

Continuing in accordance with this aspect, the first augment or the second augment may include any of a pH sensor, a temperature sensor and a pressure sensor operatively coupled to a processor of the joint implant.

### BRIEF DESCRIPTION OF THE DRAWINGS

A more complete appreciation of the subject matter of the present disclosure and the various advantages thereof can be realized by reference to the following detailed description, in which reference is made to the following accompanying drawings:

FIG. 1 is a side view of a knee joint implant according to an embodiment of the present disclosure;

FIG. 2 is a side view of a knee joint implant according to another embodiment of the present disclosure;

FIG. 3A is a side view of a tibial augment according to an embodiment of the present disclosure;

FIG. 3B is a top view of the tibial augment of FIG. 3A;

FIG. 4A is a side view of a femoral augment according to an embodiment of the present disclosure;

FIG. 4B is a top view of the femoral augment of FIG. 4A;

FIG. 5A is a side view of a femoral augment according to another embodiment of the present disclosure;

FIG. 5B is a top view of the femoral augment of FIG. 5A;

FIG. 6A is a side view of a femoral augment according to another embodiment of the present disclosure;

FIG. 6B is a top view of the femoral augment of FIG. 6A;

FIG. 7A is a side view of a femoral augment according to another embodiment of the present disclosure;

FIG. 7B is a top view of the femoral augment of FIG. 7A;

FIG. 8A is a top view of a wedge augment for an acetabular shell according to another embodiment of the present disclosure;

FIG. 8B is a side view of the wedge augment of FIG. 8A;

FIG. 9A is side view of a tibial augment according to another embodiment of the present disclosure;

FIG. 9B is a side view of a femoral augment according to another embodiment of the present disclosure;

FIG. 9C is a side view of a tibial augment according to another embodiment of the present disclosure;

FIG. 9D is a side view of a femoral augment according to another embodiment of the present disclosure;

FIG. 10A is a perspective view of a glenoid augment according to another embodiment of the present disclosure;

FIG. 10B is a perspective view of a glenoid augment according to another embodiment of the present disclosure;

FIG. 10C is a perspective view of a glenoid augment according to another embodiment of the present disclosure;

FIG. 11A is a side view of a glenoid augment according to another embodiment of the present disclosure;

FIG. 11B is a side view of a glenoid augment according to another embodiment of the present disclosure;

FIG. 11C is a side view of a glenoid augment according to another embodiment of the present disclosure, and

FIG. 12 is a side view of a glenoid augment according to another embodiment of the present disclosure.

### DETAILED DESCRIPTION

Reference will now be made in detail to the various embodiments of the present disclosure illustrated in the accompanying drawings. Wherever possible, the same or like reference numbers will be used throughout the drawings to refer to the same or like features within a different series of numbers (e.g., 100-series, 200-series, etc.). It should be noted that the drawings are in simplified form and are not drawn to precise scale. Additionally, the term “a,” as used in the specification, means “at least one.” The terminology includes the words above specifically mentioned, derivatives thereof, and words of similar import. Although at least two variations are described herein, other variations may include aspects described herein combined in any suitable manner having combinations of all or some of the aspects described.

As used herein, the terms “load” and “force” will be used interchangeably and as such, unless otherwise stated, the explicit use of either term is inclusive of the other term. Similarly, the terms “magnetic markers” and “markers” will be used interchangeably and as such, unless otherwise stated, the explicit use of either term is inclusive of the other term.

In describing preferred embodiments of the disclosure, reference will be made to directional nomenclature used in describing the human body. It is noted that this nomenclature is used only for convenience and that it is not intended to be limiting with respect to the scope of the present disclosure. As used herein, when referring to bones or other parts of the body, the term “anterior” means toward the front part of the body or the face, and the term “posterior” means toward the back of the body. The term “medial” means toward the midline of the body, and the term “lateral” means away from the midline of the body. The term “superior” means closer to the head, and the term “inferior” means more distant from the head.

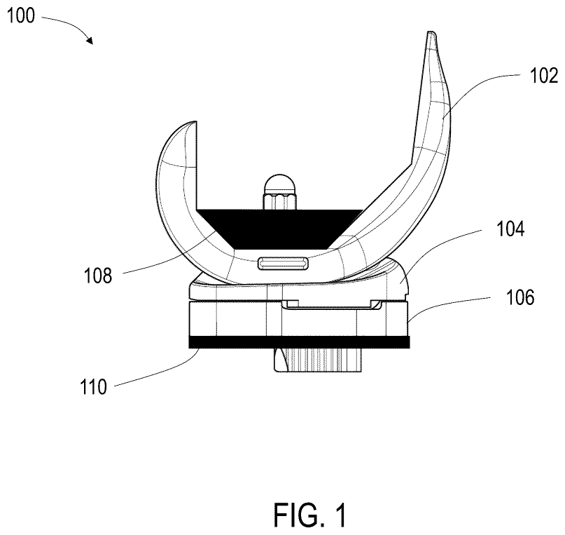

FIG. 1 is front view of a knee joint implant 100 according to an embodiment of the present disclosure. Knee joint implant 100 includes a femoral implant 102, a tibial baseplate or tibial implant 106 and a tibial insert 104 positioned between these two implants. Femoral implant 102 and tibial implant 106 are configured to be compatible with augments for difficult primary or revision cases. For example, a femoral augment 108 is coupled to femoral implant 102 and a tibial augment 110 coupled to tibial implant 106. Femoral augment 108 can include a medial femoral augment and a lateral femoral augment. Similarly, tibial augment 110 can include a medial tibial augment and a lateral tibial augment. Patients undergoing primary knee replacement, including partial knee arthroplasty (“PKA”) or TKA, often require revision surgeries due to mechanical failures, infections, aseptic loosening, joint instability, or progressive disease affecting the joint. These conditions may lead to deteriorating bone quality and weakening of ligaments over time. Revision surgeries frequently involve significant bone loss from removing previous implants, often exacerbated by the underlying disease, leading to substantial condylar defects. Surgeons address these defects by resecting the affected bone, potentially more extensively on one condyle, creating an offset between the condylar surfaces. To realign the joint and ensure a secure fit, a femoral augment is attached to the bone-facing side of the femoral component to bridge the gap between the femoral component and the resected surfaces, effectively increasing the thickness of the prosthesis to compensate for the bone deficiency.

Femoral augment 108 and tibial augment 110 can be constructed as solid metal pieces, featuring central openings designed either for securing with a screw in femoral augments or accommodating the delta keel in tibial augments. These augments, which can be as small as 5 mm, serve to fill bone gaps that extend beyond the standard resection boundaries typically used in primary knee implant surgeries. Given that these augments are made from solid metal yet are not the primary load-bearing components of the implant, they present an opportune location for integrating sensors and other electronic circuitry. The augments include a hollowed-out section specifically for housing various sensors and associated electronics. Various sensors are included in femoral augment 108 and tibial augment 110 to track implant condition, knee range of motion, patient recovery, etc. For example, encoder tracks can be integrated into both the medial and lateral femoral augments. These tracks can consist of various structures, such as magnetic tape of different lengths and magnetic markers placed at specific intervals. The resolution of the encoder track is adjustable to match the precision needed for measuring parameters like joint displacement, rotation, and slip. Each medial and lateral tibial augment includes a corresponding read head-medial and lateral, respectively-to detect magnetic flux density from the tracks. These read heads can be any type of suitable magnetometer, such as a Hall effect sensor, configured to detect and measure magnetic flux density. As the tibia rotates relative to the femur during knee movements like flexion and extension, the encoder tracks move past their respective read heads. This movement alters the magnetic flux density, a change that the read heads detect and use to monitor various knee joint implant functions including movement, rotation, speed, range of articulation, and slip.

The magnetic-mechanic coupling between the read heads and the encoder tracks of the augments facilitates direct, instantaneous, and continuous measurement of these parameters. A data transmitter, such as an antenna on one of the augments, wirelessly transmits the measured knee joint implant parameters to external devices like smartphones, tablets, monitors, or networks via Bluetooth or similar technologies. This transmission allows for real-time monitoring of knee joint implant performance. The augments can include load sensors Thus, knee joint implant 100 can simultaneously provide knee motion information (rotation, speed, flexion angle, etc.) and knee load (medial load, medial load center, lateral load, lateral load center, etc.) in real time to an external source.

Knee joint implant 100 includes a battery within either the femoral or tibial augment to power sensors and enable data transmission to external devices for real-time monitoring. This battery can be a solid-state battery, lithium-ion battery, lithium carbon monofluoride battery, lithium thionyl chloride battery, lithium-ion polymer battery, etc. Additionally, various sensors can be included in the augments, further enhancing the functionality of the implant by providing comprehensive monitoring capabilities. These sensors may include, but are not limited to, an inertial measurement unit (IMU), which can be utilized either singularly or in pairs to monitor the orientation and motion of the implant. An accelerometer, also available in singular or paired configurations, can be employed to detect changes in velocity, providing critical data on the movement and activity levels of the implant. A Hall/magnet pair can be integrated to measure magnetic fields, which could be useful in tracking the relative positioning of components or detecting shifts in the implant. A pH sensor can be incorporated to monitor the local chemical environment, potentially providing early warnings of infection or other complications. Pressure or force sensors can be used to assess the load-bearing status of the implant, offering insights into stress distribution and identifying potential points of failure. A vibration sensor could serve multiple roles, such as detecting falls or monitoring for signs of implant loosening, which are critical for the early intervention and maintenance of implant stability. Additionally, biophoton or other light sensors can be utilized for advanced monitoring of biological processes, such as tissue growth or oxygenation levels, further enhancing the diagnostic and therapeutic potential of the implant system.

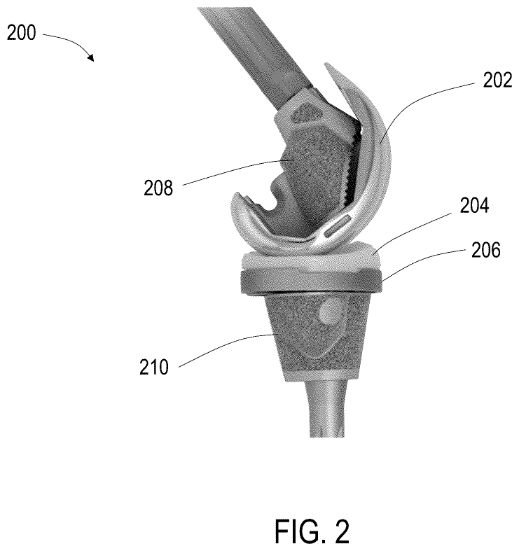

FIG. 2 is a side view of a knee joint implant 200 according to another embodiment of the present disclosure. Knee joint implant 200 is similar to knee joint implant 100, and therefore like elements are referred to with similar numerals within the 200-series of numbers. For example, knee joint implant 200 includes a femoral implant 202, a tibial implant 206 and a tibial insert 204. However, knee joint implant 200 includes a cone-shaped femoral augment 208 and a cone-shaped tibial augment 210.

The femoral and tibial augments can include components such as a printed circuit board assembly, a battery, Hall sensors, and a charging coil. All components, both electronic and non-electronic, are strategically positioned away from the load centers, which correspond to areas of high loading. These components are located in regions within the augments that experience minimal loading and forces, enhancing their measurement capabilities. The electronic components, such as the printed circuit board, are designed in a modular format like a sensor card, making them easily insertable into the augments. This modular design simplifies the maintenance and replacement of the various components of the knee joint. For instance, a surgeon can perform an in-office or outpatient procedure to access the tibia below the patella, an area with minimal tissue disruption, allowing for quick recovery to reach the sensor card in tibial augment 210. The design is such that these electronic components and sensors are modular, facilitating the easy replacement or upgrading of the augments without affecting the femoral implant or the tibial stem.

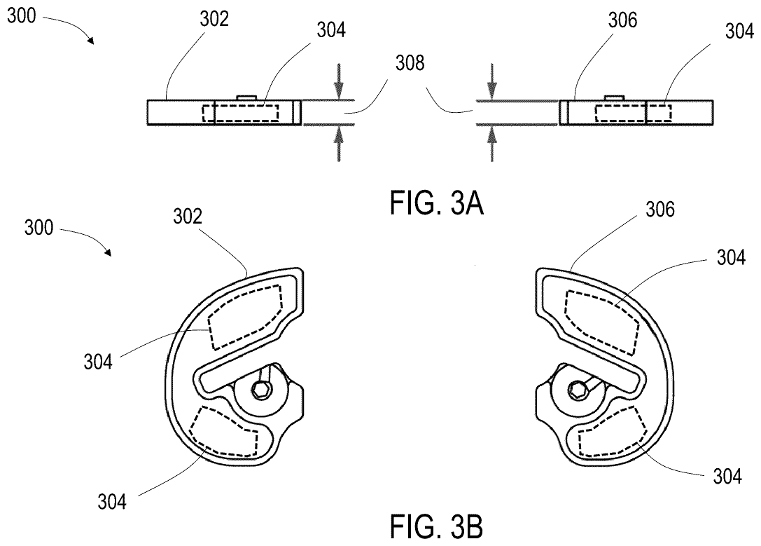

FIGS. 3A and 3B show a tibial augment 300 including a medial tibial augment 302 and a lateral tibial augment 306 according to an embodiment of the present disclosure. Tibial augment 300 incorporates sensor assemblies 304 at various positions on both the medial and lateral tibial augments as best shown in FIG. 3B. Tibial augment 300, constructed from solid metal and not serving as the primary load-bearing components of the implant, offers an optimal site for embedding sensors and other electronic circuitry. Tibial augment 300 includes hollow sections specifically designed to house sensor cards or sensor assemblies 304. Sensor card 304 can be equipped with a variety of components including a battery, communication module, inertial measurement unit (IMU), pressure sensor, temperature sensor, Hall effect sensor, or other relevant sensors. The augment can include a magnet to interact with a Hall effect sensor, enhancing functionality. The positioning of sensors within the joint space is particularly advantageous for infection detection, as the only separation between the sensor card and the joint space is the wall of the augment. Moreover, the placement of augments is strategically valuable for capturing pressure data from both the medial and lateral compartments of the femur and tibia. This enables detailed studies to assess the impact of measuring pressure on the bone-facing side of the implant versus the articulating side, ensuring that the data gathered is clinically relevant and useful for patient care.

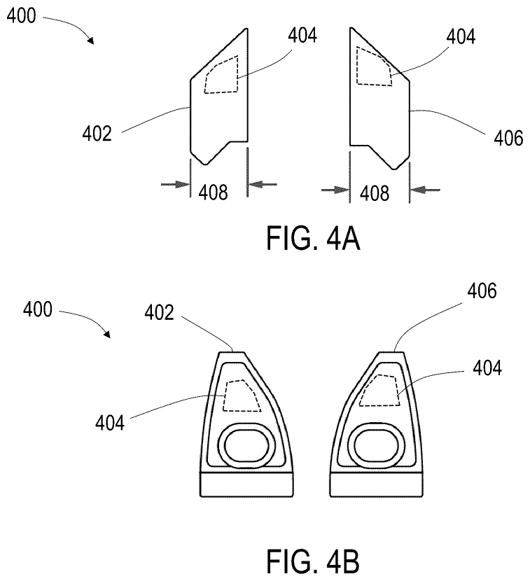

Referring now to FIGS. 4A and 4B, there is shown a femoral augment 400 comprising a medial femoral augment 402 and a lateral femoral augment 406, in accordance with an embodiment of the present disclosure. Femoral augment 400 integrates sensor assemblies 404 at various locations on both the medial and lateral femoral augments, as depicted in FIG. 4B. Similar to tibial augments described above, femoral augment 400 is composed of solid metal and does not serve as the primary load-bearing elements of the implant. Thus, femoral augment 400 provides an ideal location for embedding sensors and electronic circuitry.

Femoral augment 400 is designed with hollow sections specifically engineered to accommodate sensor cards or sensor assemblies 404. These sensor cards can be outfitted with a variety of components, such as a battery for power, a communication module for data transmission, an inertial measurement unit (IMU) for motion tracking, a pressure sensor for monitoring load, a temperature sensor for detecting changes in temperature, a Hall effect sensor for measuring magnetic fields from a corresponding tibial augment, and other relevant sensors. The strategic placement of sensors within the joint space offers significant advantages, particularly for measurement of joint implant performance, implant condition and joint condition.

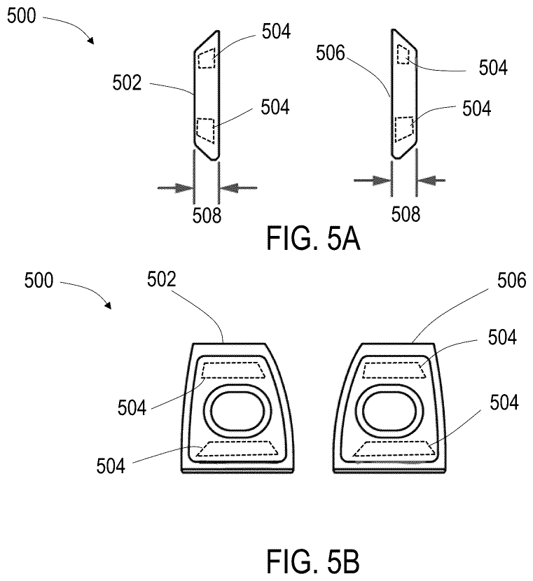

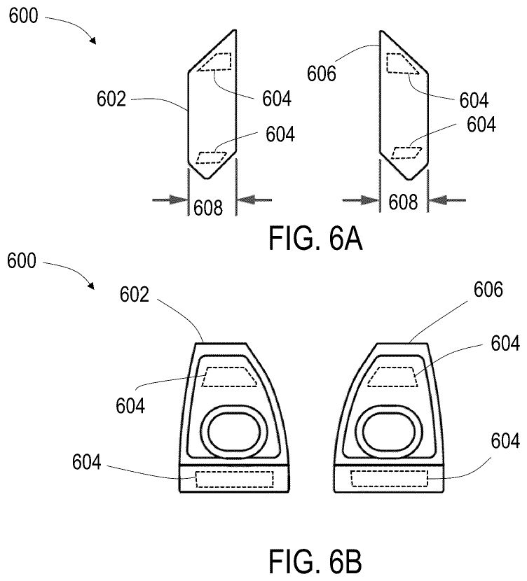

FIGS. 5A and 5B show a femoral augment 500 according to another embodiment of the present disclosure. Femoral augment 500 is similar to femoral augment 400, and therefore like elements are referred to with similar numerals within the 500-series of numbers. For example, femoral augment 500 includes a medial femoral augment 502 and a lateral femoral augment 506 with sensor cards or assemblies 504. Similarly, FIGS. 6A and 6B show a femoral augment 600 according to another embodiment of the present disclosure. Femoral augment 600 is similar to femoral augment 400, and therefore like elements are referred to with similar numerals within the 600-series of numbers.

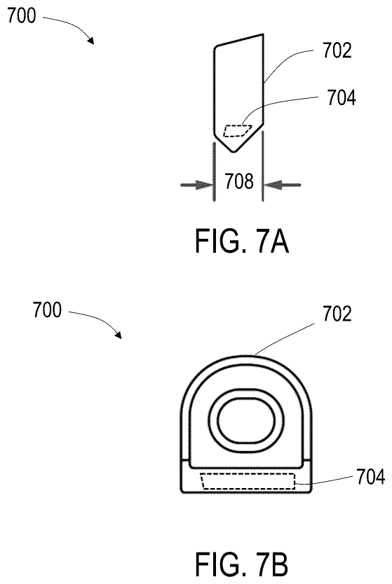

A femoral augment 700 according to another embodiment of the present disclosure is shown in FIGS. 7A and 7B. Femoral augment 700 is similar to femoral augment 400, and therefore like elements are referred to with similar numerals within the 700-series of numbers. However, femoral augment 700 includes a single sensor card 704 integrated with femoral augment body 702 as best shown in FIG. 7B.

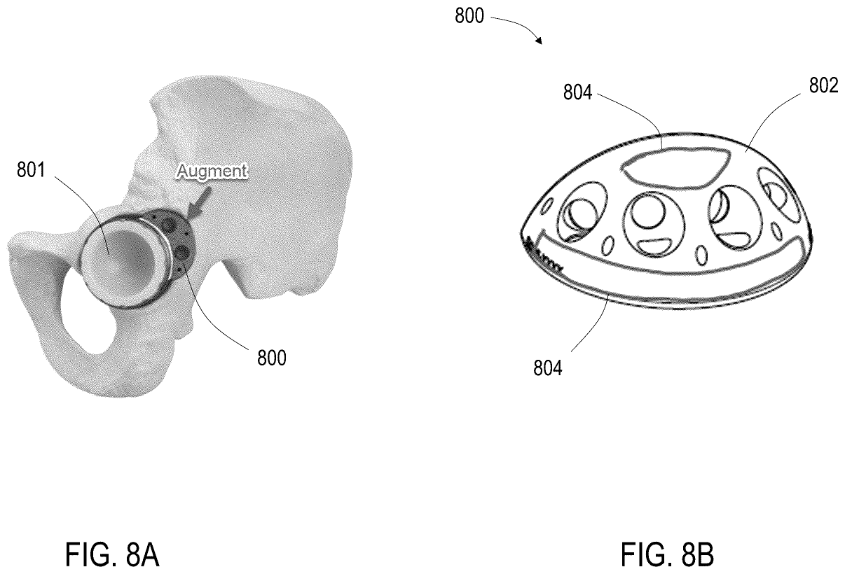

FIGS. 8A and 8B show an acetabular augment 800 according to another embodiment of the present disclosure. Acetabular augment 800 is depicted as a restoration wedge augment seated in a tritanium shell 801 in this embodiment, as best shown in FIG. 8A. The tritanium shell provides a robust and biocompatible structure, ensuring secure fixation and stability within the acetabulum. Acetabular augment 800 is similar in structure to the femoral augment 400, and therefore, similar elements are designated with numerals within the 800 series for consistency and clarity. For instance, acetabular augment 800 includes a acetabular body 802, and sensor cards 804. Multiple sensor cards 804 are integrated within the body of acetabular augment 800, as most clearly illustrated in FIG. 8B. These sensors are designed to monitor various parameters such as load distribution, temperature, and positional stability, providing real-time feedback to the healthcare provider as more fully described above. The sensor cards are strategically embedded within the body of the wedge-shaped augment to ensure accurate data collection without compromising the structural integrity of the augment. The wedge-shaped acetabular augment 800 is specifically engineered to be seated in an acetabular defect. The design allows for optimal fit and support within the defect, promoting proper anatomical alignment and facilitating the restoration of joint function.

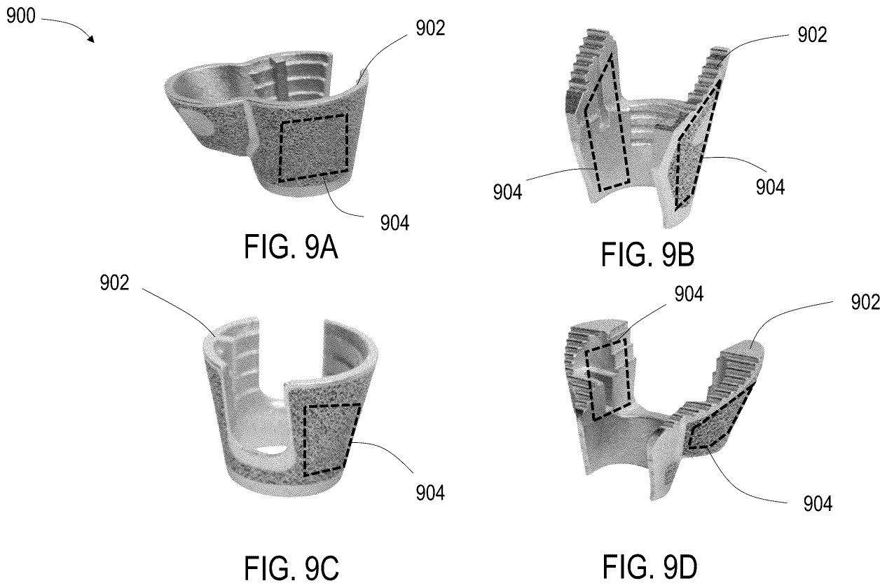

Referring now to FIGS. 9A-9D, there are shown femoral and tibial augments 900 according to other embodiments of the present disclosure. Femoral and tibial augments 900 are similar to femoral augment 400, and therefore, similar elements are designated with numerals within the 900-series for clarity and consistency. Femoral and tibial augments 900 are cone-shaped augments configured for use in revision implant procedures, such as the revision knee joint implant 200 shown in FIG. 2. The cone-shaped design provides a secure and stable fit within the femoral cavity, ensuring proper alignment and support during the revision procedure. Sensor cards 904 are integrated within the existing or thickened walls of the cone implant body 901. As described above, these sensor cards are strategically embedded to monitor various parameters such as stress distribution, temperature, and positional stability. The cone-shaped implant body 901 of femoral and tibial augments 900 is designed to provide optimal load distribution and support, enhancing the longevity and performance of the revision implant. The integration of sensor technology within the thickened walls of the augment body ensures that data collection does not compromise the structural integrity of the implant. Femoral and tibial augments 900 not only facilitate anatomical restoration during revision implant procedures but also enhance postoperative monitoring through the integration of advanced sensor technology by providing continuous, real-time data to support clinical decision-making and ensure the long-term success of the implant.

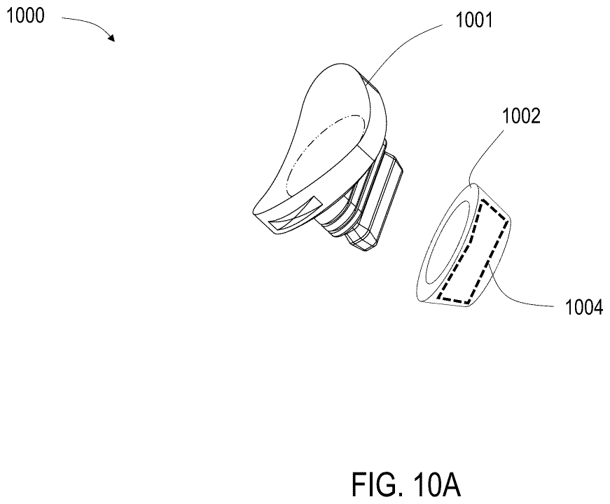

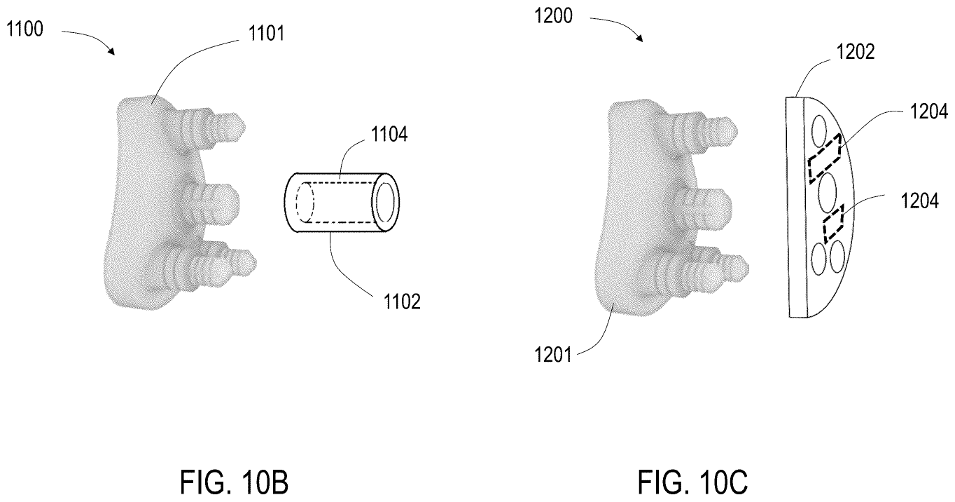

FIG. 10A shows a glenoid augment 1000 according to another embodiment of the present disclosure. Glenoid augment 1000 is configured to be placed in a glenoid defect, providing structural support, and facilitating anatomical restoration. Glenoid augment 1000 includes an augment body 1002 and an integrated sensor card 1004. Augment body 1002 is designed to couple with various glenoid implants, including a glenoid sleeve, curve plate, peg, keel, etc. Augment body 1002 is shaped as a sleeve that couples to a keeled glenoid 1001 in FIG. 10A. FIG. 10B shows a glenoid augment 1100 with an augment body 1102 shaped as a sleeve that couples to a pegged glenoid 1101 according to another embodiment. Sensor cards 1104 are integrated within walls of augment body 1102. Another embodiment shown in FIG. 10C shows a glenoid augment 1200 including a plate-shaped augment body 1202 that couples to a pegged glenoid 1201. Multiple sensor cards 1204 are integrated within augment body 1202. This versatility allows the augment to be used in a range of surgical scenarios, enhancing its applicability and effectiveness. Sensor cards are strategically embedded to monitor critical parameters such as load distribution, temperature, and positional stability, providing real-time data to the healthcare provider. The placement of the sensor card within the thickened wall ensures accurate data collection while maintaining the structural integrity and durability of the augment.

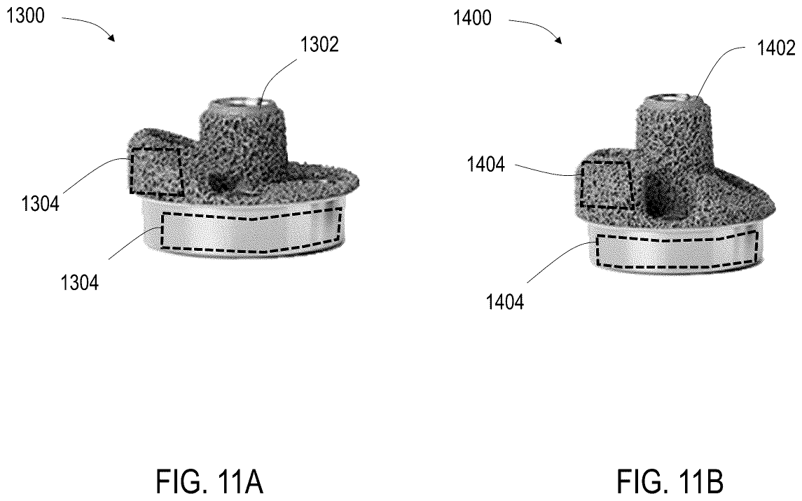

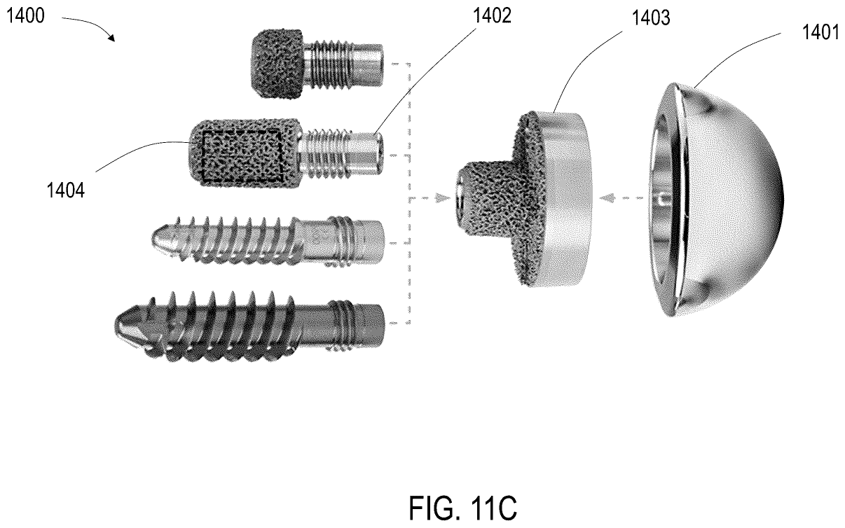

FIGS. 11A-11C show glenoid augments for a reversed glenoid implant, according to other embodiments of the present disclosure. These glenoid augments are designed to be placed in a glenoid defect and include integrated sensors within the augment body for enhanced postoperative monitoring. Glenoid augments can be integrated into a reversed base plate, with the augment bodies available in either wedge-shaped and/or base plate-shaped configurations. For instance, FIG. 11A shows a glenoid augment 1300 that includes a glenoid body 1302 with an integrated sensor card 1304. In this embodiment, the sensor card is integrated within the base plate. Additionally, there is a second sensor card 1304 embedded within the wedge-shaped portion of the augment body, providing comprehensive monitoring capabilities. Similarly, FIG. 11B illustrates another embodiment of a glenoid augment 1400 with an augment body 1402. This embodiment also features a sensor card integrated into the base plate and another sensor card within the wedge-shaped portion of the body. These sensor cards are strategically placed to monitor critical parameters such as load distribution, temperature, and positional stability, ensuring accurate data collection and real-time feedback.

FIG. 11C shows another embodiment of a glenoid augment 1400 according to the present disclosure. Augment body 1402 is a post that can be coupled to a reversed glenoid implant 1401 via a base 1403. A sensor card 1404 is integrated into augment body 1402 as shown in FIG. 11C. As more fully described above, sensor cards in these augments are designed to provide real-time data to the healthcare provider, facilitating improved clinical decision-making and patient outcomes. The augment bodies are constructed from biocompatible material, ensuring durability and safe interaction with the surrounding biological tissues. The design of these augments allows for a secure fit within the glenoid defect, promoting proper anatomical alignment and joint function.

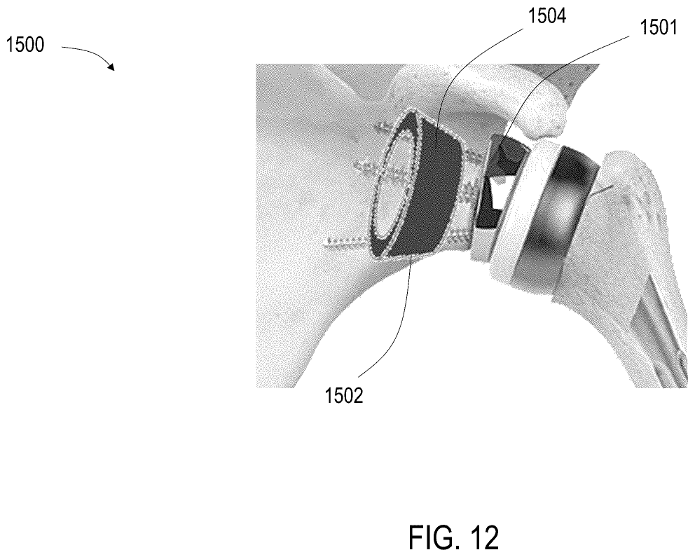

FIG. 12 shows a glenoid augment 1500 according to another embodiment of the present disclosure. Glenoid augment 1500 consists of an augment body 1502 with integrated sensor cards 1504. These sensor cards are embedded within the walls of the augment body 1502, enabling advanced monitoring capabilities. Augment body 1502 is configured to couple with a reversed glenoid implant 1501, as shown in FIG. 12. This design ensures that the augment is securely attached to the reversed glenoid implant, providing stability and proper anatomical alignment within the glenoid cavity. The glenoid augment 1500 is intended to be placed in a glenoid defect, addressing both the need for structural support and enhanced monitoring. The integration of sensor cards 1504 within the walls of the augment body allows for precise data collection on critical parameters, such as load distribution, temperature, and positional stability. This real-time data can be used for postoperative monitoring and informed clinical decision-making.

A method for monitoring the performance of a joint implant such as knee joint implant 100 is disclosed as an embodiment of the present disclosure. It should be understood that while this method is discussed with reference to knee joint implant 100 as an example, it can be applied to any joint implant. The method can include several steps designed to ensure accurate tracking and assessment of the knee joint implant's function over time. The method begins with the coupling of femoral augment 108 to a femoral implant 102 within the joint. Femoral augment is equipped with at least one magnetic marker. Subsequently, tibial augment 110 is coupled to tibial implant 106 of the joint. The tibial implant is designed to contact the femoral implant. Tibial augment 110 includes at least one magnetic sensor capable of detecting the magnetic flux density emanating from the magnetic marker on femoral augment 108. By tracking the magnetic flux density magnitudes over time using the magnetic sensor, the method enables continuous monitoring of the joint implant's performance. If the tracked magnetic flux density magnitude deviates from a predetermined value, a warning is initiated, indicating a potential issue with knee joint implant 100. The femoral augment can include both a medial femoral augment and a lateral femoral augment (FIGS. 4A and 4B), providing comprehensive coverage and monitoring capabilities across different sections of the femoral implant. The tibial augment may include a medial tibial augment and a lateral tibial augment (FIGS. 3A and 3B), ensuring detailed tracking across the tibial component. The femoral and/or tibial augment can include a pH sensor, a temperature sensor, and a pressure sensor, all operatively coupled to a processor within the knee joint implant. This enhancement allows for a more holistic monitoring approach, capturing various physiological parameters that could impact the performance and health of the joint implant. Thus, this method provides a robust framework for monitoring joint implant performance by leveraging magnetic markers and sensors ensuring accurate, real-time tracking of implant interactions, facilitating early detection of potential issues, and contributing to improved patient outcomes.

All or any of the aspects of the present disclosure can be used with any implant such as a hip implant, shoulder implant, a spinal implant, an intramedullary nail, a bone plate, a bone screw, an external fixation device, an interference screw, etc. While sensors disclosed above are generally located in the femoral and tibial augments of the knee joint implant, the sensors can be located within the femoral implant in other embodiments. Sensor shape, size and configuration can be customized based on the type of implant and patient-specific needs.

Furthermore, although the invention disclosed herein has been described with reference to particular features, it is to be understood that these features are merely illustrative of the principles and applications of the present invention. It is therefore to be understood that numerous modifications, including changes in the sizes of the various features described herein, may be made to the illustrative embodiments and that other arrangements may be devised without departing from the spirit and scope of the present invention. In this regard, the present invention encompasses numerous additional features in addition to those specific features set forth in the paragraphs below. Moreover, the foregoing disclosure should be taken by way of illustration rather than by way of limitation as the present invention is defined in the examples of the numbered paragraphs, which describe features in accordance with various embodiments of the invention, set forth in the paragraphs below.

1. A joint implant comprising:
a first augment coupled to a first implant of a joint, the first augment including at least one marker;
a second augment coupled to a second implant of the joint, the second implant contacting the first implant, the second augment including:
at least one marker reader to detect a position of the marker to identify positional data of the first augment with respect to the second augment, and
at least one load sensor to measure load data between the first and second implants; and
a processor operatively coupled to the marker reader and the load sensor,
wherein the processor outputs the positional data and the load data to an external source.

2. The joint implant of claim 1, wherein the first augment is a femoral augment and the first implant is a femoral implant.

3. The joint implant of claim 2, wherein the second augment is a tibial augment and the second implant is a tibial implant.

4. The joint implant of claim 3, wherein the tibial implant includes a tibial insert and a tibial baseplate.

5. The joint implant of claim 4, wherein the femoral augment includes a medial femoral augment and a lateral femoral augment.

6. The joint implant of claim 5, wherein the tibial augment includes a medial tibial augment and a lateral tibial augment.

7. The joint implant of claim 1, wherein the marker is a magnet and the marker reader is a magnetic sensor.

8. The joint implant of claim 7, wherein the magnetic sensor is a Hall sensor assembly including at least one Hall sensor.

9. The joint implant of claim 8, wherein the magnet is a magnetic track disposed along a surface of the first implant.

10. The joint implant of claim 1, wherein the positional data includes any of a knee flexion angle, knee varus-valgus rotation, knee internal-external rotation, knee medial-lateral translation, superior-inferior translation, anterior-posterior translation, and time derivatives thereof.

11. The joint implant of claim 1, wherein the load data includes any of a medial load magnitude, lateral load magnitude, medial load center and lateral load center.

12. The joint implant of claim 1, wherein the first augment or the second augment includes any of a pH sensor, a temperature sensor and a pressure sensor operatively coupled to the processor.

13. The joint implant of claim 1, further including a transmitter to transmit the positional data and the load data to an external source.

14. The joint implant of claim 13, wherein the external source is any of a tablet, computer, smart phone, and remote workstation.

15. A method for monitoring a joint implant performance, the method comprising the steps of:
coupling a first augment to a first implant of a joint, the first augment including at least one magnetic marker;
coupling a second augment to a second implant of the joint, the second implant configured to contact the first implant, the second augment including at least one magnetic sensor to detect a magnetic flux density of the magnetic marker;
tracking magnetic flux density magnitudes over time using the magnetic sensor, and
initiating a warning when a tracked magnetic flux density magnitude is different from a predetermined value.

16. The method of claim 15, wherein the step of coupling the first augment includes coupling a femoral augment to a femoral implant.

17. The method of claim 16, wherein the step of coupling the second augment includes coupling a tibial augment to a tibial implant.

18. The method of claim 17, wherein the femoral augment includes a medial femoral augment and a lateral femoral augment.

19. The method of claim 18, wherein the tibial augment includes a medial tibial augment and a lateral tibial augment.

20. The method of claim 15, wherein the first augment or the second augment includes any of a pH sensor, a temperature sensor and a pressure sensor operatively coupled to a processor of the joint implant.
